(19) United States
(12) Patent Application Publication
(10) Pub. No.: US 2026/0140097 A1
(43) Pub. Date: May 21, 2026

(54) SYSTEM AND METHOD FOR ANALYZING CARBON ISOTOPES OF CARBON DIOXIDE IN INCLUSIONS

(71) Applicants: BEIJING URBAN CONSTRUCTION EXPLORATION & SURVEYING DESIGN RESEARCH INSTITUTE CO., LTD, Beijing (CN); Inner Mongolia University of Technology, Hohhot (CN); Hebei University of Engineering, Handan (CN)

(72) Inventors: Xiangguo GUO, Hohhot (CN); Jianyang FENG, Beijing (CN); Shougang ZHANG, Hohhot (CN); Zhu LI, Changji (CN); Guohui ZHANG, Hohhot (CN); Shanming ZHANG, Hohhot (CN); Tianlong JIAO, Xingyi (CN)

(73) Assignees: BEIJING URBAN CONSTRUCTION EXPLORATION & SURVEYING DESIGN RESEARCH INSTITUTE CO., LTD, Beijing (CN); Inner Mongolia University of Technology, Hohhot (CN); Hebei University of Engineering, Handan (CN)

(21) Appl. No.: 19/382,334

(22) Filed: Nov. 7, 2025

(30) Foreign Application Priority Data
Nov. 20, 2024 (CN) 202411657951.6

(51) Int. Cl. G01N 30/72; G01N 1/22; G01N 1/28; G01N 1/40; G01N 33/00; H01J 49/00; H01J 49/04

(52) U.S. Cl. CPC G01N 30/72; G01N 1/2294; G01N 1/286; G01N 1/4022; G01N 1/405; G01N 33/0016; G01N 33/004; H01J 49/0031; H01J 49/0422; H01J 49/049; H01J 49/0495; G01N 2001/2866

(57) ABSTRACT

A system and method for analyzing carbon isotopes of carbon dioxide in inclusions are provided. The system includes: a carrier, a sealing cover, a gas inlet device, a flat-grinding device, a support plate, a flat-grinding driving device, a telescopic rod, an exhaust device, a first heating plate, a carbon dioxide collecting tube, a liquid nitrogen cup, a first electromagnetic three-way valve, and an isotope mass spectrometer. A flat-grinding chamber is arranged on the carrier, and the sealing cover is sealed at a top end of the carrier. The telescopic rods are positioned on two sides of the carrier, and the support plate is positioned at top ends of the telescopic rods. The flat-grinding device extends into the flat-grinding chamber. The first heating plate is mounted at a bottom end of the flat-grinding chamber.

### CROSS REFERENCE TO THE RELATED APPLICATIONS

This application is based upon and claims priority to Chinese Patent Application No. 202411657951.6, filed on Nov. 20, 2024, the entire contents of which are incorporated herein by reference.

### TECHNICAL FIELD

The present invention belongs to the field of geological sample analysis, and particularly relates to a system and method for analyzing carbon isotopes of carbon dioxide in inclusions.

### BACKGROUND

During mineral growth, fluids trapped within minerals preserve various geological and geochemical information of the geological environment at that time. The compositional constituents in the fluid inclusions are codes for interpreting the relevant geological processes. Therefore, the systematic research on the fluid inclusions enables acquisition of information regarding temperature and pressure conditions, chemical composition, and fluid sources during diagenesis and mineralization processes, for determining the evolution and function of geological fluids and explaining the processes and conditions of diagenesis and mineralization. Carbon dioxide is a relatively common component in fluid inclusions and is usually preserved in gaseous and liquid phases. Research on the carbon isotope composition of carbon dioxide in the fluid inclusions may provide important information and evidence for exploring the sources of diagenetic and metallogenic materials, the genesis of ore deposits, the physicochemical conditions of ore formation, the formation of oil and gas, the establishment of metallogenic models, and the guidance of mineral and oil-gas exploration. The vacuum crushing method is the primary method for extracting carbon dioxide from mineral inclusions.

In the prior art, the sample is first heated to 200° C. in a vacuum tube, and helium gas is introduced to purge adsorbed gases from the device walls and mineral sample surfaces, as well as gases from secondary inclusions, thereby reducing interference from carbon sources on the sample surfaces. Subsequently, the sample is heated to 600° C., causing the inclusions to burst and release gases and liquids, after which carbon dioxide is collected from the released fluids for isotope analysis. Meanwhile, since only a small amount of sample is used in the experiment, the amount of carbon dioxide obtained from the inclusions is very limited, and the influence of non-inclusion carbon sources on the experimental results is correspondingly increased. Therefore, removal of non-inclusion carbon sources is extremely important. However, in the prior art, merely preheating the sample to purge surface-adsorbed non-inclusion carbon sources is not sufficient to achieve thorough removal.

### SUMMARY

In view of this, the present invention provides a system and method for analyzing carbon isotopes of carbon dioxide in inclusions, which is used to solve the above problems.

To achieve the above objective, the present application adopts the following technical solutions.

A system for analyzing carbon isotopes of carbon dioxide in inclusions includes a carrier, a sealing cover, a gas inlet device, a flat-grinding device, a support plate, a flat-grinding driving device, a telescopic rod, an exhaust device, a first heating plate, a carbon dioxide collecting tube, a liquid nitrogen cup, a first electromagnetic three-way valve, and a mass spectrometer; where a flat-grinding chamber is arranged on the carrier, and the sealing cover is mounted at a top end of the carrier and seals the flat-grinding chamber; a pair of the telescopic rods are positioned on two sides of the carrier, and the support plate is mounted at output ends of the telescopic rods; the flat-grinding device and the flat-grinding driving device are mounted on the support plate, and an output end of the flat-grinding device passes through the sealing cover and extends into the flat-grinding chamber; the first heating plate is mounted at a bottom end of the flat-grinding chamber, two sides of the flat-grinding chamber are provided with a gas inlet and a gas outlet, the gas inlet device is in communication with the gas inlet, the exhaust device is in communication with the gas outlet, the exhaust device, the carbon dioxide collecting tube, the first electromagnetic three-way valve, and the mass spectrometer are sequentially in communication, and the carbon dioxide collecting tube is positioned in the liquid nitrogen cup.

Further, the flat-grinding device includes a flat-grinding plate, a vertical rod, and a second gear, the flat-grinding driving device includes a first motor and a first gear, the vertical rod is fixed to a top end of the flat-grinding plate, and the flat-grinding plate is positioned in the flat-grinding chamber; the vertical rod passes through the sealing cover and the support plate and is rotatably connected to the sealing cover and the support plate; and the second gear is mounted at a top end of the vertical rod, the first motor is mounted on the support plate, the first gear is mounted at an output end of the first motor, and the first gear is engaged with the second gear.

Further, a plurality of flat-grinding chambers are arranged on the carrier, two sides of each flat-grinding chamber are provided with a gas inlet and a gas outlet, a plurality of the gas inlet devices and a plurality of the flat-grinding devices are provided, numbers of the gas inlet devices and the flat-grinding devices are identical to a number of the gas inlets, the plurality of gas inlet devices are respectively in communication with the gas inlets, and the exhaust device is simultaneously in communication with the plurality of gas outlets; and the first motor is positioned at one end of the support plate, the first gear at a top end of the first motor is engaged with the second gear in an adjacent flat-grinding device, and the second gears in the plurality of flat-grinding devices are sequentially engaged with each other.

Further, the gas inlet device includes a gas inlet pipe and a first electromagnetic valve, the first electromagnetic valve is mounted in the gas inlet pipe, and the gas inlet pipe is in communication with the gas inlet.

Further, the exhaust device includes a plurality of gas treatment devices and a gas collecting pipe, a number of the gas treatment devices is identical to a number of the gas outlets, each gas treatment device includes a first gas outlet pipe, a second electromagnetic three-way valve, a second gas outlet pipe, a third gas outlet pipe, a first gas treatment trap, and a second gas treatment trap, one end of the first gas outlet pipe is in communication with the gas outlet, and another end of the first gas outlet pipe is in communication with the second electromagnetic three-way valve, the second gas outlet pipe and the third gas outlet pipe are respectively in communication with two output ends of the second electromagnetic three-way valve, the first gas treatment trap is mounted on the second gas outlet pipe, the second gas treatment trap is mounted on the third gas outlet pipe, a plurality of third gas outlet pipes are all in communication with the gas collecting pipe, and the gas collecting pipe is in communication with the carbon dioxide collecting tube.

Further, the system further includes a second heating plate, and the second heating plate is mounted at a bottom end of the flat-grinding plate.

A method for using the system for analyzing carbon isotopes of carbon dioxide in inclusions includes the following steps:
S1. opening the sealing cover and enabling the flat-grinding plate to separate from the flat-grinding chamber, placing an equal amount of inclusion samples in each flat-grinding chamber, allowing the flat-grinding plate to re-enter the flat-grinding chamber, and sealing the flat-grinding chamber with the sealing cover;
S2. lowering the telescopic rods to allow the second heating plate at the bottom end of the flat-grinding plate to descend until contacting the samples without applying pressure, driving the first motor, and flat-grinding the inclusion samples by the second heating plate in each flat-grinding chamber through transmission among the first gear, the second gear, and the engagement among the plurality of second gears;
S3. after flat-grinding for a period of time, opening the first electromagnetic valve to introduce helium gas into the flat-grinding chamber, and simultaneously opening a flow path between the second electromagnetic three-way valve and the second gas outlet pipe; fully releasing gases adsorbed on surfaces of the inclusion samples and discharging excessive air inside the device; discharging the gases into the atmosphere after harmful gases are removed through the first gas treatment trap;
S4. after a period of time, closing the flow path between the second electromagnetic three-way valve and the second gas outlet pipe, and simultaneously opening a flow path between the second electromagnetic three-way valve and the third gas outlet pipe; introducing helium gas to purge the second gas treatment trap, the gas collecting pipe, and the carbon dioxide collecting tube; and discharging gases through the first electromagnetic three-way valve into the atmosphere;
S5. closing the first electromagnetic valve and the second electromagnetic three-way valve, electrically heating the first heating plate and the second heating plate, and performing high-temperature heating on upper and lower surfaces of the inclusions until the inclusions burst and gases inside the inclusions are released;
S6. opening the first electromagnetic valve, the second electromagnetic three-way valve, and the flow path of the third gas outlet pipe, introducing helium gas so that air from the inclusions passes through the second gas treatment trap to remove water vapor, collecting gases in the gas collecting pipe and transferring the gases into the carbon dioxide collecting tube, introducing liquid nitrogen into the liquid nitrogen cup, solidifying the collected carbon dioxide under a low-temperature environment, and discharging other gases into the atmosphere through the first electromagnetic three-way valve; and
S7. removing the liquid nitrogen cup, continuously introducing helium gas, sublimating the solidified carbon dioxide into gaseous carbon dioxide again, and introducing the gaseous carbon dioxide through the first electromagnetic three-way valve into the mass spectrometer for isotope analysis and determination.

The beneficial effects of the present invention are as follows.

According to the present invention, the telescopic rods move the flat-grinding plate or the second heating plate at the bottom end of the flat-grinding plate downward in the flat-grinding chamber to contact the inclusion samples, the first motor drives the first gear to rotate, and through transmission among the first gear, the second gear, and the engagement among the plurality of second gears, flat-grinding of the inclusions is achieved. Because the plates only contact the samples without actual grinding, gases adsorbed on the surfaces of the inclusions can be fully released, while gases inside the inclusions are not released. Gases from the inclusions enter the second gas treatment trap to remove water vapor. The dried gases are collected in the carbon dioxide collecting tube and the liquid nitrogen cup, where carbon dioxide condenses first and other gases are discharged. Subsequently, the carbon dioxide is sublimated and introduced into the mass spectrometer. A plurality of flat-grinding chambers are provided to perform simultaneous flat-grinding, so that the amount of carbon dioxide collected is increased, thereby improving the accuracy of carbon isotope analysis and determination of the carbon dioxide in the inclusions.

### BRIEF DESCRIPTION OF THE DRAWINGS

To more clearly illustrate technical solutions in embodiments of the present invention or in the prior art, the following briefly introduces accompanying drawings required in the embodiments or the prior art. It is clear that the accompanying drawings in the following descriptions are only embodiments of the present invention, and a person of ordinary skill in the art may still derive other drawings from the accompanying drawings without creative efforts.

FIG. 1 is a schematic structural diagram of a carrier, a flat-grinding device, a flat-grinding driving device, a support plate, and a telescopic rod.

FIG. 2 is a top view of a carrier, a flat-grinding device, a flat-grinding driving device, a support plate, and a telescopic rod.

FIG. 3 is a sectional view taken along line A-A in FIG. 2.

FIG. 4 is a sectional view taken along line B-B in FIG. 2.

FIG. 5 is a schematic structural diagram of a carrier.

FIG. 6 is a schematic diagram of a system for analyzing carbon isotopes of carbon dioxide in inclusions.

Description of reference numerals:

10. carrier; 11. flat-grinding chamber; 111. gas inlet; 112. gas outlet; 20. sealing cover; 30. gas inlet device; 31. gas inlet pipe; 32. first electromagnetic valve; 40. flat-grinding device; 41. flat-grinding plate; 42. vertical rod; 43. second gear; 50. support plate; 60. flat-grinding driving device; 61. first motor; 62. first gear; 70. telescopic rod; 81. gas treatment device; 811. first gas outlet pipe; 812. second electromagnetic three-way valve; 813. second gas outlet pipe; 814. third gas outlet pipe; 815. first gas treatment trap; 816. second gas treatment trap; 82. gas collecting pipe; 90. first heating plate; 100. carbon dioxide collecting tube; 110. liquid nitrogen cup; 120. first electromagnetic three-way valve; 130. mass spectrometer; and 140. second heating plate.

### DETAILED DESCRIPTION OF THE EMBODIMENTS

The following clearly and completely describes the technical solutions in the embodiments of the present invention with reference to drawings in the embodiments of the present invention. It is clear that the described embodiments are merely a part rather than all of the embodiments of the present invention. All other embodiments obtained by those of ordinary skill in the art based on embodiments of the present invention without creative efforts shall fall within the protection scope of the present invention.

### Embodiment 1

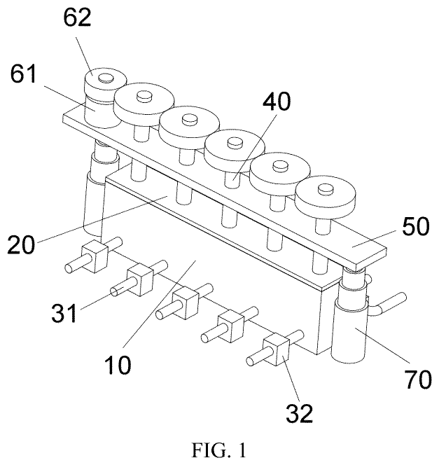

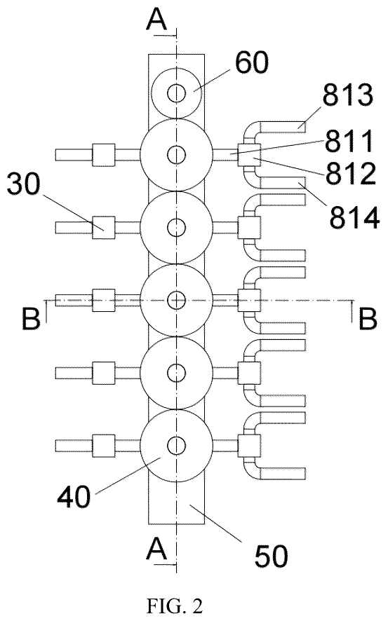

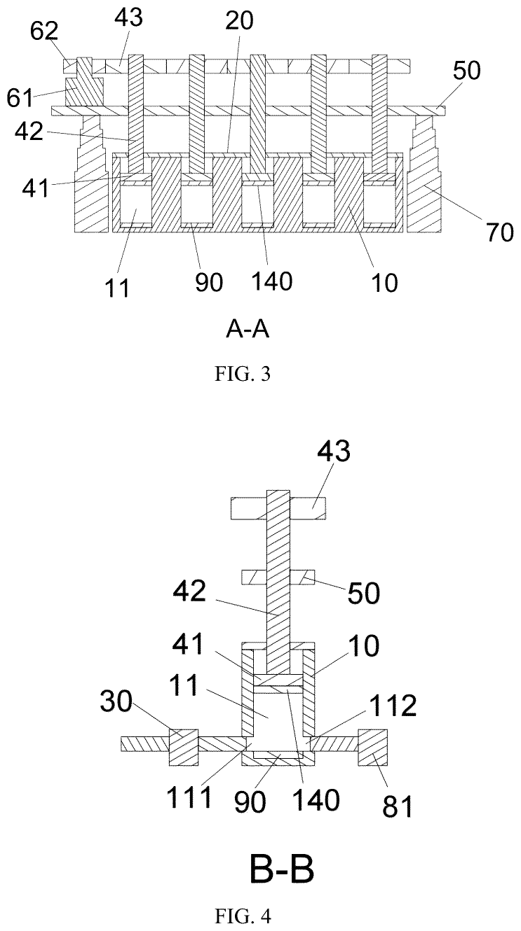

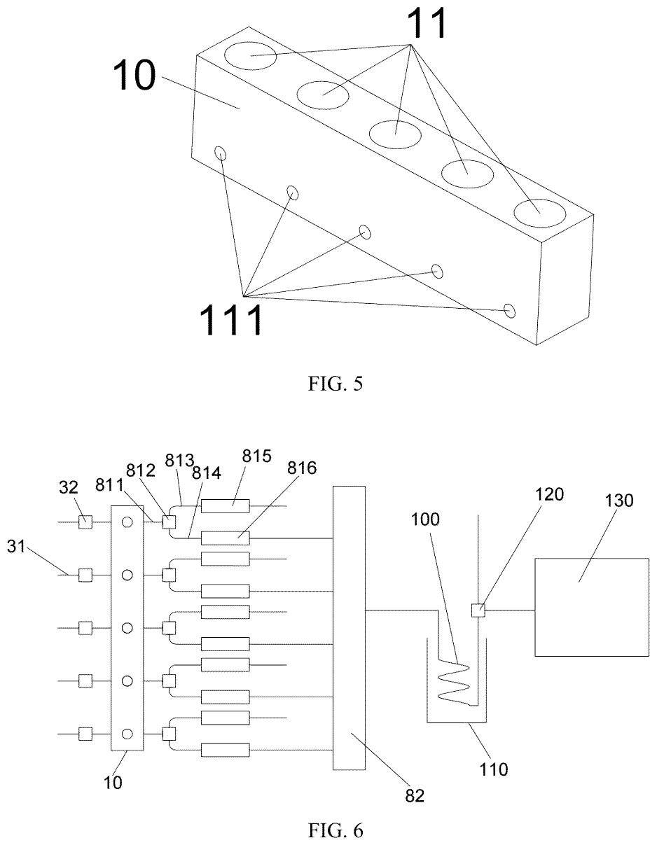

Referring to FIGS. 1-6, the present invention provides a system for analyzing carbon isotopes of carbon dioxide in inclusions, which includes a carrier 10, a sealing cover 20, a gas inlet device 30, a flat-grinding device 40, a support plate 50, a flat-grinding driving device 60, a telescopic rod 70, an exhaust device, a first heating plate 90, a carbon dioxide collecting tube 100, a liquid nitrogen cup 110, a first electromagnetic three-way valve 120, and a mass spectrometer 130; where a flat-grinding chamber 11 is arranged on the carrier 10, and the sealing cover 20 is mounted at a top end of the carrier 10 and seals the flat-grinding chamber 11; a pair of the telescopic rods 70 are positioned on two sides of the carrier 10, and the support plate 50 is mounted at output ends of the telescopic rods 70; the flat-grinding device 40 and the flat-grinding driving device 60 are mounted on the support plate 50, and an output end of the flat-grinding device 40 passes through the sealing cover 20 and extends into the flat-grinding chamber 11; the first heating plate 90 is mounted at a bottom end of the flat-grinding chamber 11, two sides of the flat-grinding chamber 11 are provided with a gas inlet 111 and a gas outlet 112, the gas inlet device 30 is in communication with the gas inlet 111, the exhaust device is in communication with the gas outlet 112, the exhaust device, the carbon dioxide collecting tube 100, the first electromagnetic three-way valve 120, and the mass spectrometer 130 are sequentially in communication, and the carbon dioxide collecting tube 100 is positioned in the liquid nitrogen cup 110.

The telescopic rod 70 contracts, driving an output end of the flat-grinding device 40 to move downward in the flat-grinding chamber 11 to contact an inclusion sample, and the flat-grinding driving device 60 drives the flat-grinding device 40 to rotate, thereby achieving flat-grinding of the inclusion. Since only contact rather than grinding is performed, gas adsorbed on a surface of the inclusion can be fully released while gas inside the inclusion is not released.

In a preferred embodiment, the flat-grinding device 40 includes a flat-grinding plate 41, a vertical rod 42, and a second gear 43, the flat-grinding driving device 60 includes a first motor 61 and a first gear 62, the vertical rod 42 is fixed to a top end of the flat-grinding plate 41, and the flat-grinding plate 41 is positioned in the flat-grinding chamber 11; the vertical rod 42 passes through the sealing cover 20 and the support plate 50 and is rotatably connected to the sealing cover 20 and the support plate 50; and the second gear 43 is mounted at a top end of the vertical rod 42, the first motor 61 is mounted on the support plate 50, the first gear 62 is mounted at an output end of the first motor 61, and the first gear 62 is engaged with the second gear 43.

In a preferred embodiment, a plurality of flat-grinding chambers 11 are arranged on the carrier 10, two sides of each flat-grinding chamber 11 are provided with a gas inlet 111 and a gas outlet 112, a plurality of the gas inlet devices 30 and a plurality of the flat-grinding devices 40 are provided, numbers of the gas inlet devices 30 and the flat-grinding devices 40 are identical to a number of the gas inlets 111, the plurality of gas inlet devices 30 are respectively in communication with the gas inlets 111, and the exhaust device is simultaneously in communication with the plurality of gas outlets 112; and the first motor 61 is positioned at one end of the support plate 50, the first gear 62 at a top end of the first motor 61 is engaged with the second gear 43 in an adjacent flat-grinding device 40, and the second gears 43 in the plurality of flat-grinding devices 40 are sequentially engaged with each other. The plurality of flat-grinding chambers 11 perform flat-grinding simultaneously, which increases a collected amount of carbon dioxide.

In a preferred embodiment, the gas inlet device 30 includes a gas inlet pipe 31 and a first electromagnetic valve 32, the first electromagnetic valve 32 is mounted in the gas inlet pipe 31, and the gas inlet pipe 31 is in communication with the gas inlet 111.

In a preferred embodiment, the exhaust device includes a plurality of gas treatment devices 81 and a gas collecting pipe 82, a number of the gas treatment devices 81 is identical to a number of the gas outlets 112, each gas treatment device 81 includes a first gas outlet pipe 811, a second electromagnetic three-way valve 812, a second gas outlet pipe 813, a third gas outlet pipe 814, a first gas treatment trap 815, and a second gas treatment trap 816, one end of the first gas outlet pipe 811 is in communication with the gas outlet 112, and another end of the first gas outlet pipe is in communication with the second electromagnetic three-way valve 812, the second gas outlet pipe 813 and the third gas outlet pipe 814 are respectively in communication with two output ends of the second electromagnetic three-way valve 812, the first gas treatment trap 815 is mounted on the second gas outlet pipe 813, the second gas treatment trap 816 is mounted on the third gas outlet pipe 814, a plurality of third gas outlet pipes 814 are all in communication with the gas collecting pipe 82, and the gas collecting pipe 82 is in communication with the carbon dioxide collecting tube 100.

In a preferred embodiment, the system for analyzing carbon isotopes of carbon dioxide in inclusions further includes: a second heating plate 140, where the second heating plate 140 is mounted at a bottom end of the flat-grinding plate 41. The first heating plate 90 and the second heating plate 140 simultaneously perform high-temperature heating on a top surface and a bottom surface of an inclusion sample until the inclusion bursts, releasing gas inside the inclusion and improving an efficiency of inclusion bursting.

### Embodiment 2

A method for using the system for analyzing carbon isotopes of carbon dioxide in inclusions includes the following steps:
S1. opening the sealing cover 20 and enabling the flat-grinding plate 41 to separate from the flat-grinding chamber 11, placing an equal amount of inclusion samples in each flat-grinding chamber 11, allowing the flat-grinding plate 41 to re-enter the flat-grinding chamber 11, and sealing the flat-grinding chamber 11 with the sealing cover 20;
S2. lowering the telescopic rods 70 to allow the second heating plate 140 at the bottom end of the flat-grinding plate 41 to descend until contacting the samples without applying pressure, driving the first motor 61, and flat-grinding the inclusion samples by the second heating plate 140 in each flat-grinding chamber 11 through transmission among the first gear 62, the second gear 43, and the engagement among the plurality of second gears 43;
S3. after flat-grinding for a period of time, opening the first electromagnetic valve 32 to introduce helium gas into the flat-grinding chamber 11, and simultaneously opening a flow path between the second electromagnetic three-way valve 812 and the second gas outlet pipe 813; fully releasing gases adsorbed on surfaces of the inclusion samples and discharging excessive air inside the device; discharging the gases into the atmosphere after harmful gases are removed through the first gas treatment trap 815;
S4. after a period of time, closing the flow path between the second electromagnetic three-way valve 812 and the second gas outlet pipe 813, and simultaneously opening a flow path between the second electromagnetic three-way valve 812 and the third gas outlet pipe 814; introducing helium gas to purge the second gas treatment trap 816, the gas collecting pipe 82, and the carbon dioxide collecting tube 100; and discharging gases through the first electromagnetic three-way valve 120 into the atmosphere;
S5. closing the first electromagnetic valve 32 and the second electromagnetic three-way valve 812, electrically heating the first heating plate 90 and the second heating plate 140, and performing high-temperature heating on upper and lower surfaces of the inclusions until the inclusions burst and gases inside the inclusions are released;
S6. opening the first electromagnetic valve 32, the second electromagnetic three-way valve 812, and the flow path of the third gas outlet pipe 814, introducing helium gas so that air from the inclusions passes through the second gas treatment trap 816 to remove water vapor, collecting gases in the gas collecting pipe 82 and transferring the gases into the carbon dioxide collecting tube 100, introducing liquid nitrogen into the liquid nitrogen cup 110, solidifying the collected carbon dioxide under a low-temperature environment, and discharging other gases into the atmosphere through the first electromagnetic three-way valve 120; and
S7. removing the liquid nitrogen cup 110, continuously introducing helium gas, sublimating the solidified carbon dioxide into gaseous carbon dioxide again, and introducing the gaseous carbon dioxide through the first electromagnetic three-way valve 120 into the mass spectrometer 130 for isotope analysis and determination.

The foregoing description is only a specific embodiment of the present invention, and the common knowledge such as the known specific structure and characteristics in the embodiments are not described in detail herein. It should be noted that various changes and modifications can be made by those skilled in the art without departing from the spirit of the present invention. These changes and modifications should also be regarded as the protection scope of the present invention, and will not affect the implementation effect of the present invention and the practicality of the patent. The protection scope claimed in the present application shall be based on the content of the claims, and the specific implementation modes and other descriptions in this specification may be used to interpret the content of the claims.

The embodiments in the specification are all described in a progressive manner, and each embodiment focuses on differences from other embodiments, and portions that are the same and similar between the embodiments may be referred to each other. Since the apparatus disclosed in the embodiment corresponds to the method disclosed in the embodiment, the description is relatively simple, and reference may be made to the partial description of the method.

The foregoing descriptions of the disclosed embodiments enables a person skilled in the art to implement or use the present invention. Various modifications to these embodiments will be readily apparent to those skilled in the art, and the generic principles defined herein may be applied to other embodiments without departing from the spirit or scope of the present invention. Thus, the present invention is not intended to be limited to these embodiments shown herein but is to be accorded the broadest scope consistent with the principles and novel features disclosed herein.

1. A system for analyzing carbon isotopes of carbon dioxide in inclusions, comprising: a carrier (10), a sealing cover (20), a gas inlet device (30), a flat-grinding device (40), a support plate (50), a flat-grinding driving device (60), a telescopic rod (70), an exhaust device, a first heating plate (90), a carbon dioxide collecting tube (100), a liquid nitrogen cup (110), a first electromagnetic three-way valve (120), and a mass spectrometer (130); wherein a flat-grinding chamber (11) is arranged on the carrier (10), and the sealing cover (20) is mounted at a top end of the carrier (10) and seals the flat-grinding chamber (11); a pair of the telescopic rods (70) are positioned on two sides of the carrier (10), and the support plate (50) is mounted at output ends of the telescopic rods (70); the flat-grinding device (40) and the flat-grinding driving device (60) are mounted on the support plate (50), and an output end of the flat-grinding device (40) passes through the sealing cover (20) and extends into the flat-grinding chamber (11); the first heating plate (90) is mounted at a bottom end of the flat-grinding chamber (11), two sides of the flat-grinding chamber (11) are provided with a gas inlet (111) and a gas outlet (112), the gas inlet device (30) is in communication with the gas inlet (111), the exhaust device is in communication with the gas outlet (112), the exhaust device, the carbon dioxide collecting tube (100), the first electromagnetic three-way valve (120), and the mass spectrometer (130) are sequentially in communication, and the carbon dioxide collecting tube (100) is positioned in the liquid nitrogen cup (110);
the exhaust device comprises a plurality of gas treatment devices (81) and a gas collecting pipe (82), a number of the gas treatment devices (81) is identical to a number of the gas outlets (112), each of the gas treatment devices (81) comprises a first gas outlet pipe (811), a second electromagnetic three-way valve (812), a second gas outlet pipe (813), a third gas outlet pipe (814), a first gas treatment trap (815), and a second gas treatment trap (816), one end of the first gas outlet pipe (811) is in communication with the gas outlet (112), and another end of the first gas outlet pipe (811) is in communication with the second electromagnetic three-way valve (812), the second gas outlet pipe (813) and the third gas outlet pipe (814) are respectively in communication with two output ends of the second electromagnetic three-way valve (812), the first gas treatment trap (815) is mounted on the second gas outlet pipe (813), the second gas treatment trap (816) is mounted on the third gas outlet pipe (814), a plurality of third gas outlet pipes (814) are all in communication with the gas collecting pipe (82), and the gas collecting pipe (82) is in communication with the carbon dioxide collecting tube (100).

2. The system for analyzing the carbon isotopes of carbon dioxide in the inclusions according to claim 1, wherein the flat-grinding device (40) comprises a flat-grinding plate (41), a vertical rod (42), and a second gear (43), the flat-grinding driving device (60) comprises a first motor (61) and a first gear (62), the vertical rod (42) is fixed to a top end of the flat-grinding plate (41), and the flat-grinding plate (41) is positioned in the flat-grinding chamber (11); the vertical rod (42) passes through the sealing cover (20) and the support plate (50) and is rotatably connected to the sealing cover (20) and the support plate (50); and the second gear (43) is mounted at a top end of the vertical rod (42), the first motor (61) is mounted on the support plate (50) the first gear (62) is mounted at an output end of the first motor (61), and the first gear (62) is engaged with the second gear (43).

3. The system for analyzing the carbon isotopes of carbon dioxide in the inclusions according to claim 2, wherein a plurality of flat-grinding chambers (11) are arranged on the carrier (10), two sides of each of the flat-grinding chambers (11) are provided with the gas inlet (111) and the gas outlet (112), a plurality of the gas inlet devices (30) and a plurality of the flat-grinding devices (40) are provided, numbers of the gas inlet devices (30) and the flat-grinding devices (40) are identical to a number of the gas inlets (111), the plurality of gas inlet devices (30) are respectively in communication with the gas inlets (111), and the exhaust device is simultaneously in communication with the plurality of gas outlets (112); and the first motor (61) is positioned at one end of the support plate (50), the first gear (62) at a top end of the first motor (61) is engaged with the second gear (43) in an adjacent flat-grinding device (40), and the second gears (43) in the plurality of flat-grinding devices (40) are sequentially engaged with each other.

4. The system for analyzing the carbon isotopes of carbon dioxide in the inclusions according to claim 1, wherein the gas inlet device (30) comprises a gas inlet pipe (31) and a first electromagnetic valve (32), the first electromagnetic valve (32) is mounted in the gas inlet pipe (31), and the gas inlet pipe (31) is in communication with the gas inlet (111).

5. The system for analyzing the carbon isotopes of carbon dioxide in the inclusions according to claim 2, further comprising a second heating plate (140), wherein the second heating plate (140) is mounted at a bottom end of the flat-grinding plate (41).

6. A method for using a system for analyzing carbon isotopes of carbon dioxide in inclusions, comprising the following steps:
S1. opening a sealing cover (20) and enabling a flat-grinding plate (41) to separate from a flat-grinding chamber (11), placing an equal amount of inclusion samples in each flat-grinding chamber (11), allowing the flat-grinding plate (41) to re-enter the flat-grinding chamber (11), and sealing the flat-grinding chamber (11) with the sealing cover (20);
S2. lowering telescopic rods (70) to allow a second heating plate (140) at a bottom end of the flat-grinding plate (41) to descend until contacting the samples without applying pressure, driving a first motor (61), and flat-grinding the inclusion samples by the second heating plate (140) in each flat-grinding chamber (11) through transmission among a first gear (62), a second gear (43), and an engagement among the plurality of second gears (43);
S3. after flat-grinding for a period of time, opening a first electromagnetic valve (32) to introduce helium gas into the flat-grinding chamber (11), and simultaneously opening a flow path between a second electromagnetic three-way valve (812) and a second gas outlet pipe (813); fully releasing gases adsorbed on surfaces of the inclusion samples and discharging excessive air inside the device; discharging the gases into an atmosphere after harmful gases are removed through a first gas treatment trap (815);
S4. after a period of time, closing the flow path between the second electromagnetic three-way valve (812) and the second gas outlet pipe (813), and simultaneously opening a flow path between the second electromagnetic three-way valve (812) and a third gas outlet pipe (814); introducing helium gas to purge a second gas treatment trap (816), a gas collecting pipe (82), and a carbon dioxide collecting tube (100); and discharging gases through a first electromagnetic three-way valve (120) into the atmosphere;
S5. closing the first electromagnetic valve (32) and the second electromagnetic three-way valve (812), electrically heating a first heating plate (90) and the second heating plate (140), and performing high-temperature heating on upper and lower surfaces of the inclusions until the inclusions burst and gases inside the inclusions are released;
S6. opening the first electromagnetic valve (32), the second electromagnetic three-way valve (812), and the flow path of the third gas outlet pipe (814), introducing helium gas so that air from the inclusions passes through the second gas treatment trap (816) to remove water vapor, collecting gases in the gas collecting pipe (82) and transferring the gases into the carbon dioxide collecting tube (100), introducing liquid nitrogen into a liquid nitrogen cup (110), solidifying collected carbon dioxide under a low-temperature environment, and discharging other gases into the atmosphere through the first electromagnetic three-way valve (120); and
S7. removing the liquid nitrogen cup (110), continuously introducing helium gas, sublimating solidified carbon dioxide into gaseous carbon dioxide again, and introducing the gaseous carbon dioxide through the first electromagnetic three-way valve (120) into a mass spectrometer (130) for isotope analysis and determination.
